(19) United States
(12) Patent Application Publication
(10) Pub. No.: US 2018/0235216 A1
(43) Pub. Date: Aug. 23, 2018

(54) DISINFECTING COMPOSITION WITH HYDROGEN PEROXIDE AND PROCESS FOR DISINFECTING SURFACES

(71) Applicant: Alvin Ronlan, Pompano Beach, FL (US)

(72) Inventor: Alvin Ronlan, Pompano Beach, FL (US)

(21) Appl. No.: 15/440,468

(22) Filed: Feb. 23, 2017

(51) Int. Cl. A01N 33/12; A01N 31/02; A01N 25/04; A01N 59/00; A61L 2/22

(52) U.S. Cl. CPC A01N 33/12; A01N 31/02; A61L 2/22; A01N 59/00; A01N 25/04

(57) ABSTRACT

The present invention relates to a volatile, almost residue free hydrogen peroxide based antimicrobial composition, with enhanced biocidal efficacy, which can be applied as a penetrating and durable, fine aerosol, that has superior strength with respect to decontaminating buildings infected with bacteria, fungi, virus or fungal or bacterial spores.

### TECHNICAL FIELD

The present invention relates to a non-staining, odorless peroxide biocidal composition which has superior strength with respect to removing bio-films and fungal and bacterial and fungal and bacterial spores. The present invention is also directed to a process for disinfecting of hard surfaces.

### BACKGROUND OF THE INVENTION

A number of studies have shown that bacterial contamination is due principally to bacterial biofilms and not planktonic bacteria. Planktonic bacteria are single organisms moving in a fluid while a bacterial biofilm consists of colonies of bacteria surrounded by a mucous, glycopolysaccharide coat which is adherent to surfaces. Most of the studies of classical microbiology starting with Louis Pasteur have been performed on bacteria growing in enriched liquid culture media or on solidified enriched culture media in petri dishes. These enriched culture conditions encourage bacteria to grow in planktonic form. In nature, bacteria exist in a more hostile environment and form biofilms. One example of a biofilm is Legionella bacteria in air conditioner cooling towers which is responsible for Legionaries disease. Bacteria in biofilms are difficult to remove from contaminated areas and their removal may require as much as a 500 times higher biocide concentration than that required to remove the same bacteria in the planktonic form. Similarly molds manifest strong resistance with respect to conventional disinfecting agents. A further serious problem associated with bacteria and molds in buildings is endo- and mycotoxins, that in many cases are not affected by disinfecting agents, i.e. the microbe that produced the toxin is killed, but the toxins remain active and continue to pose a health hazard.

Treatment with various chlorine releasing agents such as sodium hypochlorite has been conventionally used for surface disinfecting. The effect of chlorine releasing—or for that matter other oxidants- on myco- and endotoxins has not been studied. However, although chlorine bleaching agents have a superior disinfecting strength, there are disadvantages including corrosiveness, providing discomfort to the user due to the peculiar odor generated by the molecular chlorine as well as concerns regarding the toxic effects of chlorine gas, depending on the method of use. Furthermore, there is the concern that chlorine bleaching agents form environmentally harmful organic chlorine compounds as by-products. Accordingly, switching to a disinfecting agent that does not use a chlorine compound is desired.

Peroxide disinfecting agents such as hydrogen peroxide, inorganic peroxides, organic peroxides, etc., have none of the disadvantages of chlorine based agents mentioned above. Hydrogen peroxide (H202) is generally considered a highly acceptable disinfecting agent from a toxicological and environmental standpoint because its decomposition and biodegradation products are oxygen and water. Disinfectant compositions containing hydrogen peroxide are widely known throughout the industry and in the prior art.

However, used independently they all have weak disinfecting strength with respect to bacterial bio-films, fungal and bacterial spores and molds, and it is difficult to remove the molds, spores and bacterial bio-films completely with them. Furthermore, some important pathogenic strains of microbes, bacteria, viruses, etc. are especially resistant to hydrogen peroxide compositions, even though the compositions may contain a high amount of hydrogen peroxide. Moreover, known hydrogen peroxide disinfectant compositions may have an effective biocidal efficiency but at a very slow rate, e.g., on the order of hours. Thus, a hydrogen peroxide composition is desired that could disinfect a wide spectrum of germs and viruses (even especially virulent or resistant ones), with a high killing power, in a short period of time.

With regard to improvements in the bleaching (disinfecting) action of peroxide bleaching agents, various proposals have been made. In Japanese Kokai Patent Application No. Sho 61[1986]-42600 in particular, the combination of glucose pentaacetate, tetraacetylethylenediamine, tetraacetylglycoluril, cyanamide, etc., as activating agent with an oxygen bleaching agent is disclosed. In Japanese Kokai Patent Application No. Sho 52[1977]-110287, a composition for fabric bleaching agent composed of a peroxide and cyanamide and/or a metal cyanamide is disclosed. In U.S. Pat. No. 3,756,774, a composition for a fabric bleaching agent with a pH of 4-7 containing organic cyanides and peroxides is disclosed. In Japanese Kokai Patent Application No. Sho 62[1987]-1790, a mold removal composition containing peroxide and silicone oil and/or emulsifier is disclosed. In U.S. Pat. No. 5,783,550, a composition for mold removal of 4-7 containing dicyandiamide, alkali hydroxides. In U.S. Pat. No. 9,538,760, a composition for disinfecting containing hydrogen peroxide or a peroxide source, an amphoteric surfactant; and an alkali metal or alkaline earth metal salt of a cyclic or heterocyclic aromatic compound comprising at least one hydroxyl group carboxylic group, or combination thereof, is disclosed.

### SUMMARY DISCLOSURE OF INVENTION

The objective of said invention is to provide for a hydrogen peroxide based, fast acting biocidal composition having superior disinfecting strength with respect to bio-films adhered to inanimate surfaces and fungal and bacterial spores.

As a result of extensive research, I have found a surprisingly high synergy effect between of hydrogen peroxide and various aryl+alkyl or dialkyl dimethyl ammonium halides and/or certain glycols or glycol ethers, and that compositions containing hydrogen peroxide, various aryl+alkyl or dialkyl dimethyl ammonium halides and/or certain glycols or glycol ethers manifest extremely high disinfecting strength with respect to bio-films and fungal and bacterial spores without causing staining, corrosion or irritating odors.

One aspect of the present invention pertains to a method for treating surfaces in whole buildings such as food processing and pharmaceutical industries to kill or reduce or retard the growth of pathogenic microorganisms such as E. coli, salmonella, campylobacter, shigella and listeria. The method according to the invention comprises contacting a surface with an effective amount of a contacting solution comprised of hydrogen peroxide and a various aryl+alkyl or dialkyl dimethyl ammonium halides dissolved in a mixture of water and a low volatile glycol or glycol ether. The contacting solution preferably is applied as a fine aerosol produced with electrical or pulse jet (thermal) fogging equipment.

The present invention provides a safe and effective method of sanitizing surfaces by removing, reducing or retarding the growth of pathogenic microorganisms and molds without the use of substances that are toxic to humans.

### BEST MODE FOR CARRYING OUT THE INVENTION

As the peroxide used as component (A) of said invention, commercially available hydrogen peroxide aqueous solutions can be used favorably.

Next, as the various aryl+alkyl or dialkyl dimethyl ammonium halides used as component (B), commercially available various aryl+alkyl or dialkyl dimethyl ammonium halides and mixtures thereof can be used.

Next, as the diol or diol ether used as component (C), commercially available diols or diol ethers and mixtures thereof can be used.

The content of the hydrogen peroxide used as component (A) in the biocidal composition is generally 0.5-60 wt %, preferably 0.5-30 wt %, and more preferably 3-20 wt %. For practicality, 4-10 wt % is most favorable. The content of aryl+alkyl or dialkyl dimethyl ammonium halides (B) is 0.5-60 wt %, preferably 0.5-30 wt %, and more preferably 0.1-5 wt %. For practicality, 0.1-2 wt % is most favorable. Aryl+alkyl or dialkyl dimethyl ammonium halides from the of view of biodegradabability and preferred from the point of view efficiency, health and safety are Didecyldimethylammonium chloride (DDAC), Didecyldimethylammonium chloride (DDAC (C8-10)), Alkyl (C12-18) dimethylbenzyl ammonium chloride (ADBAC (C12-18)), Coco alkyltrimethylammonium chloride (ATMAC/TMAC), Alkyl (C12-18) dimethylbenzyl ammonium chloride (ADBAC (C12-18)), Alkyl (C12-16) dimethylbenzyl ammonium chloride (ADBAC/BKC (C12-C16)), Alkyl (C12-C14) dimethylbenzylammonium chloride (ADBAC (C12-C14)), Alkyl (C12-C14) ethylbenzylammonium chloride (ADEBAC (C12-C14)),

If component (A) or (B) is lower than said range, the disinfecting action is low, when component (A) or (B) is greater than said range, the product becomes difficult to handle as a biocidal composition.

The solvent (C) of the biocidal composition in said invention is important for obtaining high disinfecting strength; normally it is a mixture of water and a low volatile, water compatible glycol or glycol ether such as propylene glycol or dipropylene glycol methylether, where the content of the glycol or glycol ether in water preferable is 0-90 wt %, and more preferably 10-90 wt %.

The biocidal composition of the present invention is normally manufactured by dissolving hydrogen peroxide (A) and organic peroxide (B) in water or a mixture of water and a low volatile, water compatible glycol or glycol ether (C). The content of the solvent carrier (C) used in the biocidal composition in said invention is selected from a range of 5-99 wt %, preferably 5-95 wt %, and more preferably 10-70 wt %.

In the biocidal composition of the present invention, it is preferable to add stabilizers to stabilize the hydrogen peroxide (e.g., improve its chemical and/or physical stability), such as aromatic chelating agents or aromatic radical scavengers, known to one of ordinary skill in the art.

In the biocidal composition of the present invention, it is preferable to add a surfactant such as amphoteric surfactants including, but not limited to, betaines, nonionic surfactant such as polyoxyethylene alkyl ethers, polyoxyethylene fatty acid esters, amine oxides, etc, and cationic surfactants such as aryl+alkyl or dialkyl dimethyl ammonium halides. It is preferable for the quantity of the surfactant added to be 0.05-5 wt % in the biocidal composition. By the addition of a surfactant, it is possible to further assist in the penetration of the biocidal composition with respect to the bacterial bio-film, mold or spore coating and to enhance the biocidal effect.

The biocidal composition of the present invention is used as a solution, and it is possible to disinfect spores, bacterial bio-films or molds effectively by coating with a brush or sponge, etc., or spraying with a sprayer, etc., or fogging with a pulse jet or electrical fogger on the surfaces which are coated with bacterial bio-films or molds.

By thermal fogging, the biocidal composition of this invention is dispersed in 10 micron particle sizes and the treated building is kept closed for a minimum of twenty-four (24) hours for treatment to occur.

When spray treating with the biocidal composition of this invention a contact time is basically determined by the time it takes the biocidal composition to evaporate and can vary from of about one (1) minute to about ten (10) hours. No rinsing of treated surfaces is required prior to use or post treatment.

Typical bacteria which can be disinfected with the composition of this invention include: staphylococcus aureus, staphylococcus pyogenes, streptococcus hemolyticus, streptococcus dysgalactiae, mycobacterium tuberculosis, salmonella typhosa, salmonella typhimurium, salmonella pulorum, hemophilus parasuis, clostridium perfringens, mycoplasma synoviae, mycoplasma hyopneumoniae, pasteurella multocida, klebsiella pneumoniae, staphylococcus epidermis, streptococcus agalactiae, streptococcus fecalis, listeria monocytogenis, mycobacterium tuberculosis, salmonella choleraesuis, salmonella enteritidis, pseudomonas aeruginosa, clostridium tetani, diplococcus pneumoniae, mycoplasma gallisepticum, escherichia coli, pasteurella hemolytica, alcaligenes faecalis, salmonella gallinarum, salmonella arizonae, salmonella schotimuelleri, staphylococcus hyicus, streptococcus pyogenes, haemophilus parasuis; and, bordetella bronchiseptica.

Fungus types which may be disinfected by the composition of this invention include: aspergillus fumigatus, aspergillus glacus, aspergillus nidulans, aspergillus flavus, aspergillus niger, fusarium solani; and penicillium variable.

Spore types which may be disinfected by the composition of this invention include: Bacillus anthracis, B., and (bacterial spores) and Stachybotrys, Aspergillus, Penicillium, Trichoderma and Alternaria spp. (fungal spores).

Viruses which are disinfected by this composition include: Adenoviridae (Egg Drop Syndrome), Herpetoviridae (Infectious Bovine), Rhinotracheitis (Aujeszky's Disease), Feline Herpes, Iridoviridae (African Swine Fever), Parvoviridae, (Canine Parvovirus), Poxviridae Pseudo (Cowpox), Coronaviridae (Transmissible Gastro-Enteritis), Avian Infectious Bronchitis, Canine Coronavirus, Orthomyxoviridae (Avian Influenza), Paramyxoviridae (Newcastle Disease), Distemper, Picornaviridae (Swine Vesicular Disease), Foot & Mouth Disease, Reoviridae Gumboro (IBD), Retroviridae (Maedi & Visna), AIDS.

TABLE 1
APPLICATION EXAMPLES

| COMPONENT | EXAMPLE | | | | | | | |
|---|---|---|---|---|---|---|---|---|
| | 1 | 2 | 3 | 4 | 5 | 6 | 7 | 8 |
| HYDROGEN | 2 | 0 | 0 | 2 | 2 | 2 | 2 | 2 |
| PEROXIDE, | | | | | | | | |
| wt % | | | | | | | | |
| PROPYLENE | 0 | 10 | 0 | 10 | 0 | 10 | 70 | 70 |
| GLYCOL, | | | | | | | | |
| wt % | | | | | | | | |
| DIDECYL | 0 | 0 | 0.05 | 0 | 0.05 | 0.05 | 0 | 0.05 |
| DIMETHYL | | | | | | | | |
| AMMONIUM | | | | | | | | |
| Cl | | | | | | | | |
| WATER, wt % | 9 [symbol] | 90 | 99.95 | 88 | 97.95 | 87.95 | 28 | 27.95 |
| | 9 | 10 | 11 | 12 | 13 | 14 | 15 | 16 |
| HYDROGEN | 3 | 0 | 0 | 3 | 3 | 3 | 3 | 3 |
| PEROXIDE, | | | | | | | | |
| wt % | | | | | | | | |
| PROPYLENE | 0 | 20 | 0 | 10 | 0 | 10 | 70 | 70 |
| GLYCOL, | | | | | | | | |
| wt % | | | | | | | | |
| DIDECYL | 0 | 0 | 0.1 | 0 | 0.1 | 0.1 | 0 | 0.1 |
| DIMETHYL | | | | | | | | |
| AMMONIUM | | | | | | | | |
| Cl | | | | | | | | |
| WATER, wt % | 9 [symbol] | 80 | 99.9 | 87 | 96.9 | 86.9 | 27 | 26.9 |
| | 1 [symbol] | 18 | 19 | 20 | 21 | 22 | 23 | 24 |
| HYDROGEN | 4 | 0 | 0 | 4 | 4 | 4 | 4 | 4 |
| PEROXIDE, | | | | | | | | |
| wt % | | | | | | | | |
| PROPYLENE | 0 | 70 | 0 | 10 | 0 | 10 | 70 | 70 |
| GLYCOL, | | | | | | | | |
| wt % | | | | | | | | |
| DIDECYL | 0 | 0 | 0.3 | 0 | 0.1 | 0.1 | 0 | 0.1 |
| DIMETHYL | | | | | | | | |
| AMMONIUM | | | | | | | | |
| Cl | | | | | | | | |
| WATER, wt % | 9 [symbol] | 30 | 99.7 | 86 | 95.9 | 85.9 | 26 | 25.9 |

[symbol]

indicates data missing or illegible when filed

### Evaluation of Antimicrobial Efficacy.

The exemplary formulations described in Table 1 above were evaluated for their antimicrobial efficacy against Pseudomonas aeruginosa (ATCC 15442), Escherichia coli (ATCC 10536), Staphylococcus aureus (gram-positive type pathogenic bacteria) (ATCC 6538), Enterococcus hirae (ATCC 10541), Salmonella typhimurium (gram-negative type pathogenic bacteria) (ATCC 13311), Aureobasidium pullulans (black mold) in the following manner:

### Culture Method:

The test organisms described above were transplanted individually to suitable agar culture media and incubated (at 30-35° C. and 24 hours for the bacteria cultures and 28-30° C. and 20 days for the mold culture) to develop confluent growth, and the bacterical (mold) colonies forming units (cfu) was determined (no treatment level).

### Treatment Method:

Two Petri dishes of each culture were placed in a chamber with controlled temperature (25° C.) and humidity (85% R.H.). The chamber was filled with an aerosol (average droplet size 10 micron) of the exemplary formulation (Table 1) to be tested. Formulations containing 70% propylene glycol were applied with an electrical thermo-fogger and the other formulations were applied with a so called micro-jet aerosol generator. The amount of formulation applied in all cases corresponded to approximately 20 g of formulation per square meter culture medium. The chamber was kept closed for 24 hours and the bacterial and mold cfu were determined.

### Method for Evaluation of the Biocidal Activity:

The test method for evaluation of the bactericidal activity of a composition on clean surfaces described in European Standard, EN1276:2009, 2nd ed./AC:2010 issued by the European committee for standardisation, Brussels, was adapted to this test.

European Standard, EN1276:2009, 2nd ed./AC:2010, specifies a test method and requirements for the minimum bactericidal activity of a disinfecting composition. The test is passed if the bacterial colonies forming units (cfu) are reduced from a 10⁷ cfu (initial level) to a 10² cfu (final level after contact with the disinfecting product), i.e. a 10⁵ reduction of the viability is necessary. The results obtained for the exemplary compositions in Table 1 are shown in Table 2. “+” indicates at least a 5 log reduction in cfu and “−” indicates less than a 5 log reduction in cfu

TABLE 2
EVALUATION OF BIOCIDAL EFFICACY

| TEST ORGANISM | EXAMPLE/BIOCIDAL EFFICACY | | | | | | | |
|---|---|---|---|---|---|---|---|---|
| | 1 | 2 | 3 | 4 | 5 | 6 | 7 | 8 |
| Staphylococcus | − | − | − | − | − | + | − | + |
| aureus | | | | | | | | |
| (ATCC 6538) | | | | | | | | |
| Salmonella | − | − | − | + | + | + | + | + |
| cholerasuis | | | | | | | | |
| (ATCC 10708) | | | | | | | | |
| Pseudomonas | − | − | − | − | + | + | + | + |
| aeruginosa | | | | | | | | |
| (ATCC 15442) | | | | | | | | |
| Aureobasidium | − | − | − | − | − | − | − | + |
| pullulans | | | | | | | | |
| Aspergillus flavus | − | − | − | − | − | − | − | − |
| Penicillium sp. | − | − | − | − | − | − | − | + |
| | 9 | 10 | 11 | 12 | 13 | 14 | 15 | 16 |
| Staphylococcus | − | − | − | + | + | + | + | + |
| aureus | | | | | | | | |
| (ATCC 6538) | | | | | | | | |
| Salmonella | + | − | + | + | + | + | + | + |
| cholerasuis | | | | | | | | |
| (ATCC 10708) | | | | | | | | |
| Pseudomonas | − | − | + | + | + | + | + | + |
| aeruginosa | | | | | | | | |
| (ATCC 15442) | | | | | | | | |
| Aureobasidium | − | − | + | + | − | + | + | + |
| pullulans | | | | | | | | |
| Aspergillus flavus | − | − | − | + | − | + | + | + |
| Penicillium sp. | − | − | − | + | − | + | + | + |
| | 17 | 18 | 19 | 20 | 21 | 22 | 23 | 24 |
| Staphylococcus | + | + | + | + | + | + | + | + |
| aureus | | | | | | | | |
| (ATCC 6538) | | | | | | | | |
| Salmonella | + | + | + | + | + | + | + | + |
| cholerasuis | | | | | | | | |
| (ATCC 10708) | | | | | | | | |
| Pseudomonas | + | + | + | + | + | + | + | + |
| aeruginosa | | | | | | | | |
| (ATCC 15442) | | | | | | | | |
| Aureobasidium | + | + | + | + | + | + | + | + |
| pullulans | | | | | | | | |
| Aspergillus flavus | + | + | + | + | + | + | − | + |
| Penicillium sp. | + | + | − | + | + | + | + | + |

### Evaluation of Sporocidal Efficacy.

The exemplary formulations described in Table 1 above were evaluated for their sporocidal efficacy against Bacillus globigli, B. licheniformis and B. subtilis spores (bacterial spores) and against spores of Aspergillus and Penicillium spp.

### Contamination Simulation Method:

Suspensions in sterile water of the test organisms described above were sprayed into a test chamber (10 cubic meters) kept at 22° C. containing a one (1) square meter test surface (apiece of gypsum dry wall) mounted horizontally with the paper covered side upwards. The amount of spore spray applied was adjusted to deposit 10⁶ viable spores per square centimeter of the upper (paper covered) side.

### Treatment Method:

The chamber was filled with an aerosol (average droplet size 10 micron) of the exemplary formulation (Table 1) to be tested. Formulations containing 70% propylene glycol were applied with an electrical thermo-fogger and the other formulations were applied with a so called micro-jet aerosol generator. The amount of formulation applied in all cases corresponded to approximately 20 g of formulation per square meter of test surface. The chamber was kept closed for 24 hours and the bacterial or mold spore cfu were determined.

### Method for Evaluation of the Sporocidal Activity:

The test method for evaluation of the sporocidal activity of the compositions in Table 1 is an adaption of the European Standard, EN 1276:1997 described above.

The test is passed if the bacterial colonies forming units (cfu) are reduced from a 10⁶ cfu/cm² (initial level) to a 10¹ cfu/cm² (final level after contact with the disinfecting product), i.e. a 10⁵ reduction of the viability is necessary. The results obtained for the exemplary compositions in Table 1 are shown in Table 3. “+” indicates at least a 5 log reduction in viable spore cfu and “−” indicates less than a 5 log reduction in cfu

TABLE 3
EVALUATION OF SPOROCIDAL EFFICACY

| | EXAMPLE/BIOCIDAL | | | | | | | |
|---|---|---|---|---|---|---|---|---|
| TEST ORGANISM | EFFICACY | | | | | | | |
| | 1 | 2 | 3 | 4 | 5 | 6 | 7 | 8 |
| Bacillus globigii spores | − | − | − | − | − | − | − | − |
| Bacillus licheniformis | − | − | − | − | − | − | − | − |
| spores | | | | | | | | |
| Bacillus subtilis spores | − | − | − | − | − | − | − | − |
| Aspergillus spp.spores | − | − | − | − | − | + | − | + |
| Penicillium spp.spores | − | − | − | + | + | + | + | + |
| | 9 | 10 | 11 | 12 | 13 | 14 | 15 | 16 |
| Bacillus globigii spores | − | − | − | − | − | − | − | + |
| Bacillus licheniformis | − | − | − | − | − | + | − | + |
| spores | | | | | | | | |
| Bacillus subtilis spores | − | − | − | + | + | + | + | + |
| Aspergillus spp.spores | − | − | − | − | − | + | − | + |
| Penicillium spp.spores | − | − | − | + | + | + | + | + |
| | 17 | 18 | 19 | 20 | 21 | 22 | 23 | 24 |
| Bacillus globigii spores | − | − | − | − | − | + | − | + |
| Bacillus licheniformis | − | − | − | − | − | + | − | + |
| spores | | | | | | | | |
| Bacillus subtilis spores | + | − | − | + | + | + | + | + |
| Aspergillus spp.spores | − | − | − | − | + | + | − | + |
| Penicillium spp.spores | − | − | − | + | + | + | + | + |

Examples 6, 8, 14, 15, 16, 20, 22, 23 and 24 clearly demonstrates the great synergy for biocidal activity when hydrogen peroxide is combined with a DDCA and/or a propylene glycol. Furthermore, Examples 14, 16, 22 and 23 also exhibited very fast acting biocidal effect (less than 1 minute contact time required for log 5 CFU reduction)

Tests have been carried out to establish the virucidal activity of two of the above composition (22 and 24 in Table 1) in accordance with the standard test procedures. These tests have shown the effectiveness of the composition against the following broad spectrum of viruses and viral infections when applied as described under “Treatment Method” above, which gave a 4 log reduction in virus titre:

Adenoviridae (Egg Drop Syndrome), Herpetoviridae (Infectious Bovine), Rhinotracheitis (Aujeszky's Disease), Feline Herpes, Iridoviridae (African Swine Fever), Parvoviridae, (Canine Parvovirus), Poxviridae Pseudo (Cowpox), Coronaviridae (Transmissible Gastro-Enteritis), Avian Infectious Bronchitis, Canine Coronavirus, Orthomyxoviridae (Avian Influenza), Paramyxoviridae (Newcastle Disease), Distemper, Picornaviridae (Swine Vesicular Disease), Foot & Mouth Disease, Reoviridae Gumboro (IBD), Retroviridae (Maedi & Visna), AIDS.

From the foregoing, it is to be understood that the compositions according to the invention provide excellent and surprising disinfecting and deodorant benefits to hard surfaces. Such compositions in accordance with the present inventive teaching are particularly advantageously used against known pathogenic/nuisance microorganisms commonly found in indoor environments.

While the invention is susceptible of various modifications and alternative forms, it is to be understood that specific embodiments thereof have been shown by way of examples which however are not intended to limit the invention to the particular forms disclosed; on the contrary the intention is to cover all modifications, equivalents and alternatives falling within the scope and spirit of the invention as expressed in the appended claims.

I claim:

1. A sprayable, liquid biocidal composition, comprising:
(a) 0.5-60 wt % of hydrogen peroxide;
(b) 0.01-10 wt % of quaternary ammonium compound or combination of quaternary ammonium compounds.
(c) 0-99 wt % of a water compatible glycol or glycol ether; and
said composition being characterized by very low level residue and a biocidal, activity substantially greater than that produced by like compositions based solely on hydrogen peroxide.

2. A composition according to claim 1, wherein the quaternary ammonium compound is Didecyldimethylammonium chloride (DDAC), and the total concentration of the quaternary ammonium compound is between 0.01 and 5% by weight.

3. A composition according to claim, wherein the quaternary ammonium compound is Didecyldimethylammonium chloride (DDAC (C8-10)), and the total concentration of the quaternary ammonium compound is between 0.01 and 5% by weight.

4. A composition according to claim 1, wherein the quaternary ammonium compound is Alkyl (C12-18) dimethylbenzyl ammonium chloride (ADBAC (C12-18)), and the total concentration of the the quaternary ammonium compound is between 0.01 and 5% by weight.

5. A composition according to claim 1, wherein the quaternary ammonium compound is Coco alkyltrimethylammonium chloride (ATMAC/TMAC), and the total concentration of the the quaternary ammonium compound is between 0.01 and 5% by weight

6. A composition according to claim 1, wherein the quaternary ammonium compound is Alkyl (C12-18) dimethylbenzyl ammonium chloride (ADBAC (C12-18)), and the total concentration of the quaternary ammonium compound is between 0.01 and 5% by weight

7. A composition according to claim 1, wherein the quaternary ammonium compound is Alkyl (C12-C14) dimethylbenzylammonium chloride (ADBAC (C12-C14)), and the total concentration of the the quaternary ammonium compound is between 0.01 and 5% by weight.

8. A composition according to claim 1, wherein the quaternary ammonium compound is Alkyl (C12-C14) ethylbenzylammonium chloride (ADEBAC (C12-C14)), and the total concentration of the the quaternary ammonium compound is between 0.01 and 5% by weight

9. A composition according to claim 1, wherein the water compatible glycol or glycol ether is propylene glycol and the total concentration of propylene glycol is between 1 and 70% by weight

10. A composition according to claim 1, wherein the water compatible glycol or glycol ether is dipropylene glycol and the total concentration of propylene glycol is between 1 and 70% by weight.

11. A method of disinfecting and deodorizing and/or denaturing myco- and/or endotoxins which comprises applying the disinfectant and deodorizing solution claimed in claims 1 and 9 to a surface or object requiring disinfecting or deodorizing or myco- and/or endotoxin denaturing.
